(19) United States
(12) Patent Application Publication
(10) Pub. No.: US 2011/0213350 A1
(43) Pub. Date: Sep. 1, 2011

(54) Laser Focused Ablation Apparatus and Method of Use in Revision Arthroplasty

(76) Inventors: Bertram Ezenwa, Mequon, WI (US); Robert Limoni, Green Bay, WI (US)

(21) Appl. No.: 13/037,787

(22) Filed: Mar. 1, 2011

Related U.S. Application Data
(60) Provisional application No. 61/309,162, filed on Mar. 1, 2010.

(51) Int. Cl. A61B 18/20

(52) U.S. Cl. 606/13

(57) ABSTRACT

The present invention provides and a laser focused ablation apparatus and method of use configured to ablate a cement mantle surrounding a stem of a hip prosthesis, for use during a revision arthroplasty. The apparatus comprises a laser generation system generating a laser light and a handpiece to receive the laser light. The handpiece includes an elongated main conduit terminating at a laser emission tip, the laser emission tip providing a tip and a curvature disposed between the tip and the elongated main conduit such that the laser light exits the tip at a direction oblique to a longitudinal axis of the elongated main conduit.

### CROSS REFERENCE TO RELATED APPLICATION

This application claims the benefit of U.S. provisional application 61/309,162 filed Mar. 1, 2010 and hereby incorporated by reference.

### STATEMENT REGARDING FEDERALLY SPONSORED RESEARCH OR DEVELOPMENT

1. Field of the Invention

This invention relates generally to a laser focused ablation system and methods of use, particularly for use in the ablation of orthopedic cement during revision arthroplasty.

2. Brief Description of the Prior Art

A significant percentage of all hip arthroplasties constitute revisions of previously placed hip prostheses. The revision procedure typically includes the removal of a previously placed hip prosthesis, which has failed, and replacing it with a new hip prosthesis. On average revision arthroplasty is required 8 to 10 years after a hip prosthesis has been initially placed. As life expectancy continues to rise and patients undergo primary hip replacements at an earlier age, the need for revision arthroplasty will correspondingly rise.

One of the critical steps of the revision arthroplasty is removing the failed prosthetic hip stem from the patient's femur. Bone cement is typically used to form a cement mantle between the hip stem and the femur bone, and thereby secure the stem within the femur.

Currently, three surgical methods are routinely employed for removing the hip stem. One option is to create an anterior femoral cortical window with drill holes or an osteotomy. This procedure includes removing a portion of the anterior body of the femur bone, primarily below the intertrochanteric crest. The window formation allows the failed hip stem to be exposed along its entire length and removed therefrom by prying on the stem and or mechanical abrasion or chiseling of the cement. A second option is to perform a trochanteric osteotomy (ETO), in which a distal portion of the femur body, extending below the greater trochanter is removed to allow access to a portion of the distal side of the hip stem. After the hip stem has been pried from the femur, through the distal access, the replacement hip stem may be inserted. The removed bone portion is subsequently wired back into place during the revision arthroplasty. A third option is to use a chisel during a flexible osteotomy, during which the bone structure surrounding the hip stem is removed. The chisel is used to loosen the hip stem of the ingrown implant from both the bone cement and bone tissue at the location of the cement mantle.

Each of the routine surgical methods employed to remove a failed hip stem during a revision arthroplasty include significant risks of significant damage to the bone. Both the femoral cortical window and ETO methods require removal of significant cortical bone structure and thereby reduce the load bearing or mechanical strength of the femur. The chisel method may also result in residual damage to the cortical bone of the femur. Any such reduction in femur strength may jeopardize a patient's early return to full load bearing following artificial hip revision surgery. Furthermore, collateral damage to the spongy bone tissue, i.e. cancellous bone tissue, located internal to the cortical bone tissue of the femur, may occur. As the spongy bone tissue is critical in bearing stress exerted on the femur, any damage will impede the bone's ability to return to full remodeling functions, and thereby prolong full patient recovery.

### SUMMARY OF THE INVENTION

The present invention provides a laser system that can rapidly ablate the cement mantle surrounding a failed hip prosthesis stem, during a revision arthroplasty, with minimized mechanical trauma to the bone. Use of a laser focused ablation device provides the surgeon with the ability to ablate bone cement working from the entrance aperature of the prosthesis stem along the depth of the stem without bone removal and accurate control of the ablation volume, limiting damage to both the surrounding cancellous and cortical bone tissue.

Specifically, the present invention provides an apparatus for ablation of the cement mantle surrounding a stem of a hip prosthesis, for use during a revision arthroplasty. The apparatus includes a laser generation system generating a laser light and a handpiece to receive the laser light. The handpiece includes an elongated main channel projecting beyond a hand held body portion. The elongated main channel terminates at a laser emission tip, the laser emission tip includes a tip and a curvature located between the tip and the elongated main channel such that the laser light exits the tip at a direction oblique to a longitudinal axis of the elongated main channel. The curvature allows the surgeon to direct the emitted ablation path to be directionally oriented towards the cement mantle and exterior surface of the hip prosthesis, thereby limiting contact with the surrounding bond tissue. Additionally, the tip includes a plurality of fixed orientation secondary tips designed to focus the laser light in a predetermined ablation path as it exits the tip, thereby limiting thermal dispersion into the area surrounding the target to be ablated. The target area to be ablated including an area of approximately 4 millimeters from the external surface of the failed hip prosthesis stem.

Thus it is one object of the invention to provide a means for removing a failed hip stem from a femur while minimizing collateral damage to the surrounding bone tissue, allowing the replacement prosthesis to fixate more securely.

The laser focused ablation system may include a friction guide located along the rigid elongated main channel of the device. The elongated main channel is rigid to prevent it from deflecting from the intended location during use. Furthermore, the frictional guide may interact with the either the femoral bone tissue or the external surface of the failed hip prosthesis stem to provide additional stabilizing forces.

Thus it is another object of the present invention to provide a focused ablation penetration pathway the orientation of which is accurately controlled by surgeon.

The body of the handpiece includes an orientation indicia indicative of orientation of the curvature and tip of the elongated main channel tip.

Thus it is another object of the present invention to provide the surgeon with a means of determining the orientation of the ablation path, when a portion of the device is visually obscured.

The laser focused ablation system may also include an ablation parameter window above mechanical compression strength of the bone cement, i.e. 100 MPa, and below the mechanical compression strength of cortical bone at 154 MPa.

Thus it is another object of the present invention to provide an ablation penetration pathway configured to ablate the cement mantle surrounding a failed hip stem, but not the surrounding cortical bone.

### BRIEF DESCRIPTION OF THE DRAWINGS

FIG. 1A is a perspective view of a laser focused ablation device according to the present invention;

FIG. 1B is a detailed view of a portion of the laser focused ablation device of FIG. 1A;

FIG. 2 is a diagram representing a system incorporating the laser focused ablation device of FIG. 1A;

FIG. 3 is a flow chart of a hip revision arthroplasty procedure utilizing the laser focused ablation device of FIG. 1A;

FIG. 4 is a cross sectional view of the hip revision arthroplasty procedure utilizing the laser focused ablation device of FIG. 1A;

FIG. 5 is a cross sectional view of the tip of the laser focused ablation device according to one embodiment of the present invention; and

FIG. 6 is a cross sectional view of the tip of the laser focused ablation device according to an alternative embodiment of the present invention.

### DETAILED DESCRIPTION OF THE PREFERRED EMBODIMENT

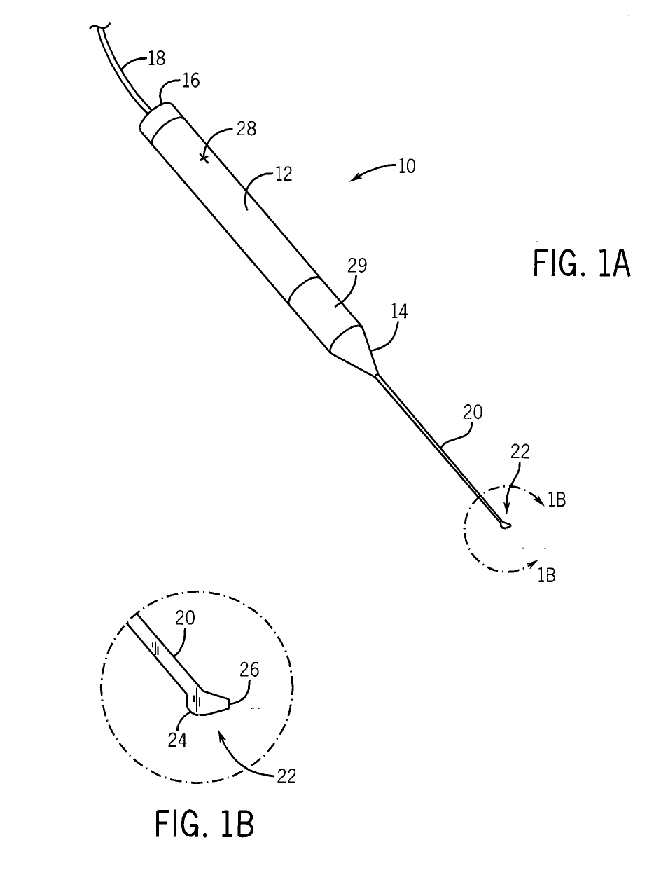

Referring initially to FIG. 1A, the present invention provides a laser focused ablation device 10. The device 10 is configured of a handpiece 12 having a first end 16 and a second end 14. The first end 16 receives a laser light along a connection means 18. The laser light is then transmitted through the handpiece 12 to the opposing side at second end 14. The elongated main conduit 20 is operably connected to the second end 14 of the handpiece, and designed to transmit the laser light therein. The elongated main conduit 20 ends at a tip 22. The tip 22 includes both a curvature 24 and a tip 26, as illustrated in greater detail in FIG. 1B.

Returning to FIG. 1A, the handpiece 12 is designed to fit comfortably within the hand of a surgeon. In one embodiment the handpiece 12 is cylindrical, and has a longitudinal length of greater than 75 millimeters and less than 250 millimeters, and a diameter of greater than 3 millimeters and less than 25 millimeters. The second end 14 may include a taper or conical structure that narrows to the point at which it is affixed to the elongated main conduit 20. The exterior surface of the handpiece 12 may include an orientation indicia 28, which is positioned to indicate the directional orientation of the curvature 24 and tip 26 of the tip 22. The orientation indicia 28 may be particularly beneficial when the elongated main conduit 20 and tip 22 are obstructed from a direct line of sight, such as when they are extended between a patient's femur and the stem of the hip prostheses during revision arthroplasty. Additionally, the exterior surface of the handpiece 12 may include a hand grip 29 adjacent the second end 14, designed to improve the surgeon's grasp of the handpiece 12. The hand grip 29 may be formed of a soft deforming gel, or a material providing improved frictional engagement with the surgeon's hand. Finally, the first end 16 is configured to receive a connection means 18, through which the laser light is transmitted. The connection means 18 may be a fiber optic cable, a plurality of fiber optic cables or any alternative embodiment sufficient to transmit a laser light therein. Furthermore, the connection means 18 may be flexible as to allow the surgeon to move the handpiece unencumbered.

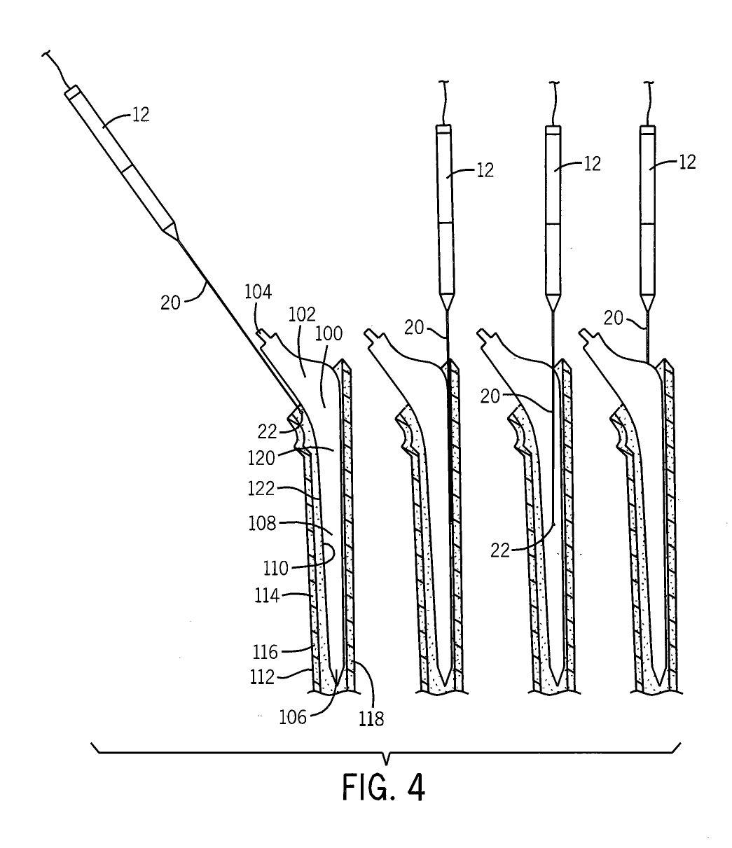

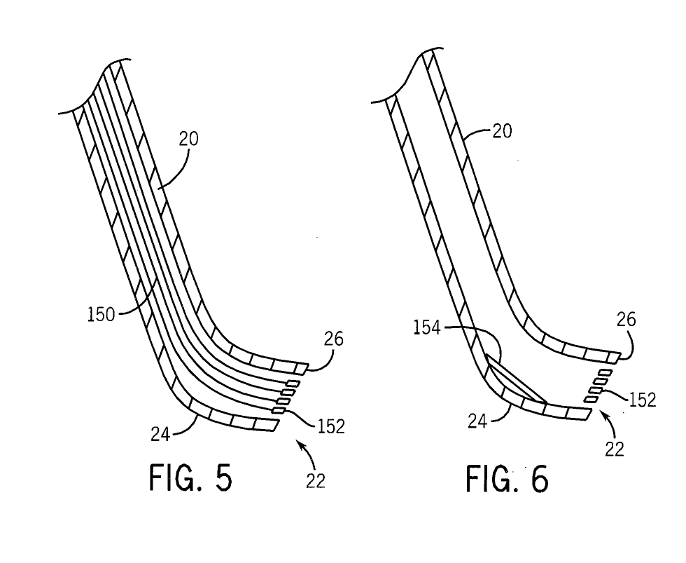

As also illustrated in FIG. 1A, the second end 14 of the handpiece 12 terminates at the elongated main conduit 20. The elongated main conduit 20 has a longitudinal length of approximately 150 millimeters and has a cross sectional width of approximately 1.5 millimeters. The elongated main conduit 20 is designed to be both long and narrow, such that it can extend into the femur adjacent the stem of a hip prosthesis during the ablation of the cement mantle. To this end the elongated main conduit 20 must be sufficiently long, as to extend along the longitudinal length of the hip stem, and narrow, as to minimize the width of the required ablation path necessary to insert the elongated main conduit 20, as depicted in FIG. 4. The elongated main conduit 20 is additionally a rigid structure, designed to allow the surgeon precise and complete control over its location during the ablation process. To provide the surgeon with additional control over the location of the elongated main conduit 20, a frictional material covers the external surface thereof. Accordingly, the frictional material will provide an increased frictional coefficient over the external surface of the elongated main conduit 20. The frictional material may be either a rough exterior surface of the elongated main conduit 20, or a soft polymer disposed along the surface of the elongated main conduit 20. Within the elongated main conduit 20, is a fiber optic cable, a plurality of fiber optic cables, or alternative means of transmitting the laser light therein. In on embodiment the elongated main channel may be hollow, providing a conduit in which the laser light travels.

Turning now to FIGS. 1B, 3, and 4, the end of the elongated main conduit 20, opposite the handpiece 12, terminates at the tip 22. The tip 22 may includes a curvature 24 operatively connected with the laser transmission functions of the elongated main conduit 20. The curvature 24 accordingly bends or otherwise alters the linear direction of the laser light. As depicted in FIG. 1B, the curvature 24 may bend the laser light in a direction oblique to the longitudinal axis of the elongated main conduit 20. The curvature 24 may achieve such a redirection of the laser light by means of a fiber optic cable 150, a mirror 154, a prism, or any other means of bending lasers as is known in the art. The tip 22 terminates at the tip 26. The tip 26 is configured to include a plurality of small anchored apertures 152 or secondary tips. These directionally fixed apertures 152 are intended to direct the laser light into a predefined ablation path, and limit the spread of the ablation path by focusing the laser light in a target area. Tip 26 has a diameter of greater than 1 millimeter and less than 10 millimeters. The effective ablation path may be restricted to an area less than 8 millimeters from the exterior surface of the hip stem. The tip 26 may include a plurality of small holes or apertures 152 in the exterior surface of the tip 22, a plurality of fiber optic cables 150, optical lenses, or any other means of directing a laser light as is known in the art.

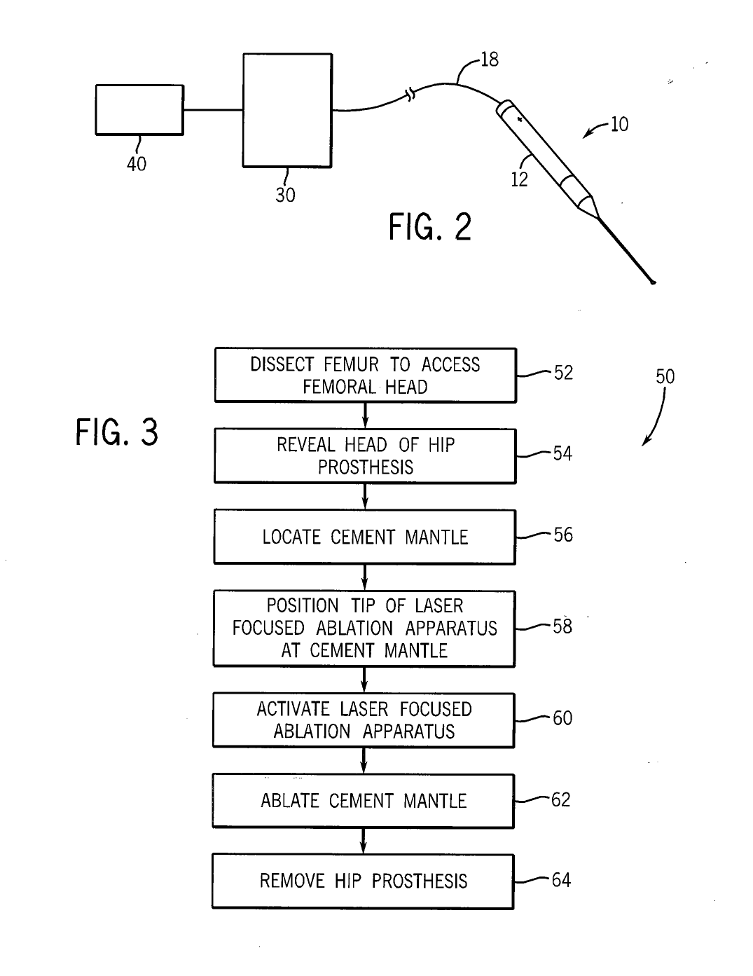

As seen in FIG. 2, the laser focused ablation device 10 is operatively connected to a laser generation system 30 by means of connection means 18. The laser generation system 30 is powered by power source 40. In one embodiment the laser generation system 30 is a solid-state lasers, utilizing a crystalline or glass rod doped with ions that provide the required energy states. In one embodiment of the present invention the laser generation system 30 included a lasing material of yttrium aluminum garnet (YAG) to which is added a dopant such as neodymium, chromium, erbium, or other ions. During operation the energy level and pulse settings of the laser generation system 30 may be altered as to maximize the ablation ability of the laser light. In one embodiment, an energy setting of 3.5 Joules and a pulse rate of 20 pulses per second resulted in a ablation power of greater than 40 Watts and less than 100 Watts. While the YAG laser is capable of producing a laser light of higher power, it was identified that blue laser pulses were ideal for the ablation of PMMA bone cement. Furthermore, the laser focused ablation device 10 of the present invention may include an ablation power that is effective only within a desired window, namely a ablation power suitable for ablating material above the mechanical compression of bonding cement, i.e. 100 MPa, and below the mechanical compression strength of cortical bone, i.e. 154 MPa.

In orthopedic surgery, poly(methyl methacrylate) (PMMA) is a thermoplastic, and used as a bone cement to affix implants and to remodel lost bone. It is supplied as a powder with liquid methyl methacrylate (MMA). When mixed these yield a cement that gradually hardens. Surgeons may judge the curing of the PMMA bone cement by pressing their thumb on it. Bone cement acts like a grout during arthroplasty. Although sticky, it does not bond to either the bone or the implant, it primarily fills the spaces between the prosthesis and the bone preventing motion. PMMA vaporizes to gaseous compounds (including its monomers) upon laser cutting, and as such allows the YAG laser to easily and accurately form an ablation cut. In one embodiment of the present invention the PMMA may be dyed to a color that provides a greater absorption of laser light, to more effectively focus the ablation path at the cement mantle.

During arthroplasty the bone cement forms a cement mantle 122 around an elongated body 108 of the hip stem 100, as seen in FIG. 4. The hip stem 100 includes an upper head 102 and a lower end 106. The upper head 102 includes a fixation means 104, for receiving a ball joint thereon. The elongated body 108 extends between the upper head 102 and lower end 106, and has an exterior surface 110. The elongated body 108 is set within the femur 112, into the cancellous bone tissue 120 between the medial side 116 of the cortical bone tissue 114 and the distal side 118 of the cortical bone tissue 114. The exterior surface 110 of the hip stem 100 elongated body 108 is thereby encased in a cement mantle 122 at the location where it interfaces with the cancellous bone tissue 120, i.e. spongy bone tissue, of the inner femur 112.

Referring to FIG. 3, during revision arthroplasty, as depicted in box 52 of flow chart 50 the femur bone is surgically dissected to gain access to a femoral head of the femur. Box 54 depicts the head of the hip prosthesis is revealed. The power source 40 provides power to the laser generation system 30. The laser generation system 30 is activated to produce a laser light. The laser light is transmitted through the connection means 18 to the laser focused ablation device 10. The surgeon grasps handpiece 12, thereby positioning the tip 22 of the elongated main conduit 20 in contact with the cement mantle 122, as seen in boxes 56 and 58 of FIG. 3. More specifically, the tip 26 is directed towards the exterior surface 110 of the hip stem 100. Accordingly, during activation of the laser, indicated in boxes 60 and 62, the ablation pathway is directed towards the cement mantle 122 and exterior surface 110, and away from the cancellous bone tissue 120 as illustrated in FIG. 4. Such an orientation limits the laser light's thermal spread to the cancellous bone tissue 120, and resultantly limits collateral damage to the femur 112. As illustrated in FIG. 4, the surgeon may relocated the laser focused ablation device 10 about the exterior surface 110 of the hip stem 100 to ablate the cement mantle 122 on all sides, including the medial, distal anterior and posterior surface as illustrated respectfully in FIG. 4. Following the ablation of the cement mantle 122, the hip stem 100 is removed from the femur 112, as depicted in box 64.

In one embodiment, the laser focused ablation device 10 is directed primarily towards the exterior surface of the hip stem 100, such that any residual ablation or thermal energy emitted by the laser light is absorbed by the hip stem 100 and does not cause damage to the surrounding cancellous bone tissue 120 or cortical bone tissue 114. In another embodiment, the laser focused ablation device 10 may be used to ablate in-growth of cancellous bone tissue 120 to the hip stem 100, while not ablating the cortical bone tissue 114 of the femur 112. In yet another embodiment, the laser focused ablation system may be used in conjunction with the formation of an anterior cortical femoral window, or alternative revision arthroplasty procedure.

A revision arthroplasty according to the present invention will accordingly result in a preserved mechanical strength of the femur 112, a decrease in the volume of cancellous bone tissue 120 damaged or removed during the removal of the failed hip stem 100, reduced surgical procedure time, and reduced patient recovery time.

The present invention has been described in terms of the preferred embodiment, and it is recognized that equivalents, alternatives, and modifications, aside from those expressly stated, are possible and within the scope of the appending claims.

We claim:

1. A laser ablation apparatus adapted to ablate a cement mantle surrounding a stem of a hip prosthesis; the apparatus comprising:
a laser generation system providing a laser light;
a handpiece operatively connected to the laser generation system to receive the laser light, the handpiece providing:
a hand grip adapt for grasping by the human hand;
an elongated main conduit extending from the hand grip to receive and conduct the laser light therealong, the elongated main conduit,
a laser emission tip at an end of the elongated main channel receiving the laser light therefrom to direct the laser light from the laser emission tip in a direction oblique to the axis of the elongated main conduit,
the laser light having an ablation power of greater than 40 Watts and less than 100 Watts.

2. The laser focused ablation apparatus of claim 1, wherein the laser generation system further provides an yttrium aluminum garnet laser medium.

3. The laser focused ablation apparatus of claim 1, wherein the elongated main conduit provides a friction promoting outer surface.

4. The laser focused ablation apparatus of claim 1, wherein the elongated main conduit provides a longitudinal length of at least 75 millimeters.

5. The laser focused ablation apparatus of claim 1, wherein the elongated main conduit further includes a fiber optic therein to transmit the laser light.

6. The laser focused ablation apparatus of claim 1, wherein the elongated main conduit further provides a plurality of fiber optic elements to transmit the laser light therein.

7. The laser focused ablation apparatus of claim 1, wherein the tip provides of plurality of apertures.

8. The laser focused ablation apparatus of claim 6, wherein each of the plurality of apertures is in operative communication with one of a plurality of fiber optic cables disposed within the elongated main conduit.

9. The laser focused ablation apparatus of claim 6, wherein the tip has a diameter of less than eight millimeters.

10. The laser focused ablation apparatus of claim 1, wherein the laser tip includes a mirror to direct the laser light at the direction oblique to a longitudinal axis of the elongated main conduit.

11. The laser focused ablation apparatus of claim 1, providing a direction indicia on the handpiece to indicate an orientation of the direction of the tip.

12. The laser focused ablation apparatus of claim 1, wherein the laser light is adapted to ablate the cement mantle.

13. The laser focused ablation apparatus of claim 1, wherein cement mantle is formed of poly(methyl methacrylate).

14. A method of ablating a cement mantle surrounding a hip prosthesis stem located within the femur of a patient; the method providing the steps of:
surgically dissecting the femur of a patient to gain access to a femoral head of the femur;
revealing the hip prosthesis located within the femur;
locating the cement mantle disposed at a outer circumference of the hip prosthesis stem, between an exterior surface of the hip prosthesis stem and a cancellous tissue;
positioning a tip of a laser focused ablation apparatus in contact with the cement mantle, the tip further comprising a tip oriented to direct a laser ablation path towards the exterior surface of the hip prosthesis stem;
activating the laser focused ablation apparatus to emit a laser ablation path and ablate the cement mantle;
moving the tip of a laser focused ablation apparatus about the outer circumference of the hip prosthesis stem; and
removing the hip prosthesis stem from the femur of a patient.

15. The method of claim 14, wherein the step of activating the laser focused ablation apparatus further provides the steps of:
activating a yttrium aluminum garnet laser generating system to produce a laser light; and
transmitting the laser light from the yttrium aluminum garnet laser generating system to a handpiece.

16. The method of claim 15, further providing the steps of:
transmitting the laser light along plurality of fiber optic cables disposed within an elongated main conduit extending from the handpiece.

17. The method of claim 16, further providing the steps of:
emitting the laser light from the tip, wherein the tip comprises a plurality of anchored outlets, wherein each of the plurality of anchored outlets is in operative communication with one of a plurality of fiber optic cables disposed within the elongated main conduit.

18. The method of claim 14, wherein the step of activating the laser focused ablation apparatus to emit a laser ablation path and ablate the cement mantle further provides the steps of:
emitting the laser ablation path in a target area, the target area located within 4 millimeters of the outer circumference of the hip prosthesis stem.

19. The method of claim 14, wherein the step of activating the laser focused ablation apparatus to emit a laser ablation path and ablate the cement mantle further provides the steps of:
vaporizing the cement, wherein the cement is formed of poly(methyl methacrylate).

20. The method of claim 12, further providing the steps of:
limiting an ablation power of the laser light such that it may ablate compounds having a mechanical compression strength of greater than 100 MPa and less than 154 MPa.
